(19) United States
(12) Patent Application Publication
(10) Pub. No.: US 2016/0184246 A1
(43) Pub. Date: Jun. 30, 2016

(54) TRANSDERMAL DRUG DELIVERY SYSTEMS FOR AGOMELATINE

(71) Applicant: Noven Pharmaceuticals, Inc., Miami, FL (US)

(72) Inventors: Puchun Liu, Miami, FL (US); Patrick Dayal, Miami, FL (US); Steven Dinh, Miami, FL (US)

(73) Assignee: Noven Pharmaceuticals, Inc., Miami, FL (US)

(21) Appl. No.: 14/982,395

(22) Filed: Dec. 29, 2015

Related U.S. Application Data
(60) Provisional application No. 62/097,932, filed on Dec. 30, 2014.

(51) Int. Cl. A61K 31/165; A61K 9/70; A61K 9/00

(52) U.S. Cl. CPC A61K 31/165; A61K 9/0014; A61K 9/7084

(57) ABSTRACT

Described are transdermal drug delivery systems for the transdermal administration of agomelatine. Methods of making and using such systems also are described.

### CROSS-REFERENCE TO RELATED PATENT APPLICATIONS

This application claims priority under 35 U.S.C. §119(e) to U.S. provisional application 62/097,932, filed Dec. 30, 2014, which is incorporated herein by reference in its entirety.

### FIELD OF THE INVENTION

Described herein are compositions and methods for the transdermal delivery of agomelatine. The agomelatine compositions and methods are useful, for example, in the treatment of depression.

### BACKGROUND

Transdermal delivery systems (adhesive patches) as dosage forms have been the subject of a vast number of patent applications over the last 25 years, yielding many patents but few commercial products in comparison. To those working in the field, the relatively small number of commercial products is not surprising. Although regulatory, economic, and market hurdles play a role in limiting the number of products on the market, the task of developing a transdermal delivery system that achieves desired physical and pharmacokinetic parameters to satisfy physician and patient demand is more daunting. Parameters to be considered during commercial product development may include drug solubility, drug stability (e.g., as may arise from interaction with other component materials and/or the environment), delivery of a therapeutic amount of drug at a desired delivery rate over the intended duration of use, adequate adhesion at the anatomical site of application, integrity (e.g., minimal curling, wrinkling, delaminating and slippage) with minimal discomfort, irritation and sensitization both during use and during and after removal, and minimal residual adhesive (or other components) after removal. Size also may be important from a manufacturing and patient viewpoint, and appearance may be important from a patient viewpoint.

Agomelatine is a melatonin analog being developed as an antidepressant for the treatment of depression, major depressive disorder (MOD), and mood disorders. Pharmacologically, agomelatine is a melatonin MT1 and MT2 receptor agonist and a serotonin 5-HT2C receptor antagonist, and increases levels of dopamine and noradrenaline in areas of the brain involved in mood control. See, e.g., Srinivasan et al., J. Neuropsych. Clin. Neurosci. 24: 290-308 (2012). Agomelatine promises significant benefits over other antidepressants such as paroxetine, venlafaxine, and stertraline, including: (1) reduced sexual side effects, (2) improved quality of sleep, (3) no discontinuation syndrome, and (4) generally weight neutral.

Oral dosage forms (tablets) of agomelatine have been approved in Europe under the trade names Valdoxan, Melitor, and Thymanax. Each tablet contains 25 mg agomelatine, prescribed at an initial dose of 1 tablet taken at bedtime, which dose may be increased to 2 tablets (50 mg) as needed. Oral agomelatine is well absorbed (although greater in women than in men), however, oral bioavailability is only 5% with large variations. The limited bioavailability is believed to be due to heavy first-pass liver metabolism, predominantly by cytochrome CYP1A2 of the P450 isoenzyme system, which results in conjugated metabolites of hydroxylated and demethylated agomelatine. The drug is bound by 95% to plasma protein, but rapidly eliminated with a plasma half-life (t_{1/2}) of 1-2 hours. The agomelatine oral tablet product produces a human plasma concentration-time pharmacokinetic (PK) profile with a Cmax of about 6 ng/ml, declining thereafter thereafter with a t_{1/2} of less than 2 hours.

Because of this heavy first-pass hepatic metabolism, dose-related hepatotoxicity risk of agomelatine has resulted in warning updates and monitoring guidance. Agomelatine also may cause increases in serum hepatic transaminases, as high as three times the upper limit of normal range, in up to 1% of individuals. Drugs that inhibit cytochrome CYP1A2 (e.g., fluvoxamine, estrogens, propranolol) may slow the metabolism of agomelatine, resulting in increased and variable agomelatine levels.

A transdermal dosage form of agomelatine could avoid or minimize some of these problems, such as by avoiding hepatic metabolism. Thus, there is a need for transdermal drug delivery systems designed for the delivery of agomelatine.

### SUMMARY

In accordance with some embodiments, there are provided transdermal drug delivery systems for the transdermal delivery of agomelatine in the form of a flexible finite system for topical application, comprising a composition comprising agomelatine and an enhancer. In some embodiments, the enhancer is selected from the group consisting of isopropanol and ethanol. In some embodiments, the composition comprises an amount of the enhancer effective to achieve substantially complete drug delivery within 12 hours of application or less. In some embodiments, the composition comprises an amount of the enhancer effective to achieve at least about 60% of the agomelatine delivery within about 4 to about 6 hours of application. In some embodiments, the composition comprises an amount of the enhancer effective to achieve at least about 75% of the agomelatine delivery within about 4 to about 6 hours of application. In some embodiments, the composition comprises an amount of the enhancer effective to achieve at least about 80% of the agomelatine delivery within about 6 to about 8 hours of application.

In accordance with any of the foregoing embodiments, the composition may be a drug-in-solution reservoir-type composition comprising agomelatine and aqueous isopropanol, a drug-in-gel reservoir-type composition comprising agomelatine, aqueous isopropanol, and a gelling agent, or a drug-in-polymer matrix composition comprising agomelatine formulated in a polymer matrix comprising isopropanol.

In accordance with any drug-in-solution reservoir-type embodiments or any drug-in-gel reservoir-type embodiments, the aqueous isopropanol may comprise 50-75% v/v isopropanol. In specific embodiments, the agomelatine is present at its saturation concentration. In further specific embodiments, the composition comprises at least 50 mg/mL agomelatine.

In accordance with any drug-in-polymer matrix embodiments, the polymer matrix may comprise a pressure sensitive adhesive polymer, such as an acrylic polymer, a silicone polymer, or a mixture of two or more thereof. In some embodiments, the agomelatine is present at its saturation concentration in the polymer matrix. In specific embodiments, the polymer components comprise about 70% (w/w) acrylic polymer and about 30% (w/w) silicone polymer, based on the dry weight of the polymer components. In specific embodiments, the polymer matrix composition comprises about 10.5% (w/w) agomelatine and about 6% (w/w) isopropanol.

In accordance with any embodiments described herein, the system may further comprise a backing and/or a release liner.

In accordance with other embodiments, there are provided methods of making a transdermal drug delivery system for the transdermal delivery of agomelatine, comprising preparing a drug-containing composition comprising agomelatine and an enhancer.

In accordance with other embodiments, there are provided methods of transdermally delivering agomelatine, comprising applying a transdermal drug delivery system as described herein to the skin or mucosa of a subject in need thereof. In some embodiments, the subject is suffering from depression. In some embodiments, the delivery of agomelatine is substantially completed within 12 hours or less.

In accordance with other embodiments, there are provided methods of treating depression in a subject in need thereof, comprising applying a transdermal drug delivery system as described herein once daily to the skin or mucosa of a subject in need thereof.

In accordance with other embodiments, there are provided transdermal drug delivery systems as described herein for use in treating depression.

In accordance with other embodiments, there are provided uses of agomelatine in the preparation of a transdermal drug delivery system as described herein, in the preparation of a medicament for treating depression.

### BRIEF DESCRIPTION OF THE DRAWINGS

FIG. 1A illustrates transdermal delivery system as described herein that consists of a backing, a drug-containing layer, and a release liner (when present). FIG. 1B illustrates a transdermal delivery system as described herein that further comprises a face adhesive layer between the drug-containing layer and release liner (when present). FIG. 1C illustrates a reservoir-type transdermal delivery system as described herein that includes of a backing, a reservoir, a membrane (which may be a microporous or EVA membrane or a rate-controlling membrane), a peripheral or face adhesive layer, and a release liner (when present).

FIG. 2A shows the in vitro flux through human cadaver skin of agomelatine and isopropanol from a formulation of agomelatine prepared at saturation concentration in 50% aqueous isopropanol, such as for a drug-in-solution reservoir-type transdermal drug delivery systems as described herein, and FIGS. 2B-2D shows the same from formulation of agomelatine prepared at saturation concentration in 25%, 75%, and 90% aqueous isopropanol, respectively.

FIG. 3 shows the plasma concentration in swine of agomelatine as delivered from drug-in-gel reservoir type transdermal drug delivery systems as described herein.

FIG. 4 shows the in vitro flux through human cadaver skin of agomelatine from a drug-in-polymer matrix type transdermal drug delivery systems as described herein.

### DETAILED DESCRIPTION

Transdermal drug delivery systems often are designed to provide prolonged, sustained drug delivery; however, that type of drug delivery is not optimal or suitable for all drugs. For some drugs, like agomelatine, a relatively short period of drug delivery is desired, to provide an “on-and-off” effect, e.g., where the drug effect is “on” and then shortly thereafter is “off” This type of drug delivery profiled can be difficult to achieve from transdermal drug delivery systems without removing the systems, particularly for solid drugs like agomelatine. Nevertheless, described herein are transdermal drug delivery systems for the transdermal delivery of agomelatine that achieve a relatively short period of drug delivery for a desired pharmacokinetic profile with an on-and-off effect. In some embodiments, the transdermal drug delivery systems are drug-in-solution or drug-in-gel reservoir-type systems. In other embodiments, the transdermal drug delivery systems are drug-in-polymer matrix type systems.

### DEFINITIONS

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein generally means that the described composition (e.g., transdermal drug delivery system, polymer matrix, etc.) comprises less than about 5%, less than about 3%, or less than about 1% by weight, based on the total weight of the composition at issue, of the excluded component. The phrase “free of” as used herein means that the described composition (e.g., polymer matrix, etc.) is formulated without adding the excluded component(s) as an intended component, although trace amounts may be present in other components or as a by-product or contaminant, such that the composition comprises at most only trace amounts of the excluded component(s).

As used herein “subject” denotes any animal in need of drug therapy, including humans. For example, a subject may be suffering from or at risk of developing a condition that can be treated or prevented with agomelatine, or may be taking agomelatine for health maintenance purposes. In specific embodiments, the subject is a subject suffering from depression, including major depressive order.

As used herein, the phrases “therapeutically effective amount” and “therapeutic level” mean that drug dosage or plasma concentration in a subject, respectively, that provides the specific pharmacological response for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease. In some embodiments, a therapeutically effect amount of agomelatine is about 1-3 mg/day administered transdermally, based on the 5% bioavailability of the 25 mg/day and 50 mg/day oral doses.

As used herein, “flux” (also called “permeation rate”) is defined as the absorption of a drug through skin or mucosal tissue, and is described by Fick's first law of diffusion:

J=−D(dCm/dx)

where J is the flux in g/cm²/hr, D is the diffusion coefficient of the drug through the skin or mucosa in cm²/hr and dCm/dx is the concentration gradient of the drug across the skin or mucosa.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system (e.g., a device) comprising a composition that releases drug upon application to the skin (or any other surface noted above). A transdermal drug delivery system may comprise a drug-containing composition, and, optionally, a backing layer and/or a release liner layer. In some embodiments, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. Typically, transdermal drug delivery systems are classified into one of two categories: matrix-type systems and reservoir-type systems, as discussed in more detail below.

A transdermal drug delivery system also may include a drug impermeable backing layer or film. In some embodiments, the backing layer is adjacent the drug-containing composition. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotch Pak™ 1012 or 9732 (a polyester film with an ethylene vinyl acetate copolymer heat seal layer), 9723 (a laminate of polyethylene and polyester), or CoTran 9720 (a polyethylene film) are useful in the transdermal drug delivery systems described herein, as are Dow® backing layer films, such as Dow® BLF 2050 (a multi-layer backing comprising ethylene vinyl acetate layers and an internal SARAN™ layer.

A transdermal drug delivery system also may include a release liner, typically located adjacent the opposite face of the system as compared to the backing layer. When present, the release liner is removed from the system prior to use to expose the polymer matrix layer and/or an adhesive layer prior to topical application. Materials suitable for use as release liners are well-known known in the art and include the commercially available products of Dow Corning Corporation designated Bio-Release® liner and Syl-off® 7610, Loparex's PET release liner (silicone-coated) and 3M's 1020, 1022, 9741, 9744, 9748, 9749 and 9755 Scotchpak™ (fluoropolymer-coated polyester films).

A transdermal drug delivery system may be packaged or provided in a package, such as a pouchstock material used in the prior art for transdermal drug delivery systems in general. For example, DuPont's Surlyn® can be used in a pouchstock material. Alternatively, a pouchstock comprising a coextruded ethylene acrylic acid/low-density polyethylene (EAA/LDPE) material, or Barex® from INEOS (acrylonitrile-methyl acrylate) may be used.

Transdermal Drug Delivery Systems for Agomelatine

As noted above, compositions and methods for the transdermal delivery of agomelatine offer significant advantages over oral dosage forms, including improved and more consistent bioavailability and reduced liver toxicity by avoiding hepatic metabolism. However, it is difficult to design a transdermal drug delivery system that provides a short drug delivery period with an “on-and-off” drug effect, such as may be desired for agomelatine. While some transdermal products with a short drug delivery period have been developed for liquid drugs with high skin permeability (such as amphetamine or nicotine), such systems have not been developed for solid drugs like agomelatine.

In accordance with the compositions and methods described herein, agomelatine is formulated in a composition for transdermal delivery, wherein the composition comprises an amount of enhancer effective to achieve the desired drug delivery profile. In particular, the present inventors have determined that by selecting and controlling the type and amount of enhancer, agomelatine can be formulated for transdermal delivery to achieve drug delivery over a relatively short period of time (e.g., 12 hours or less), even if the transdermal delivery system is left in place on the subject's skin for a longer period of time (e.g., for one day). This permits the design of a “once daily” system for the delivery of agomelatine, such that subjects can apply a system once a day at the same time of day (usually in the evening), and leave the system in place until about the same time the following day, even though drug delivery is to be completed in less than one day. For example, in specific embodiments, a composition is formulated such that at least about 60% of the drug delivery that will occur from the system occurs within the first 4 to 8 hours, or 4 to 6 hours, after application. In other embodiments, at least about 75% of the drug delivery that will occur from the system occurs within the first 4 to 8 hours, or 4 to 6 hours, after application. In yet other embodiments, at least about 80% of the drug delivery that will occur from the system occurs within the first 4 to 8 hours, or 4 to 6 hours, after application. In still other embodiments, at least about 80% of the drug delivery that will occur from the system occurs within the first 6 to 12 hours, or 6 to 8 hours, after application. In still other embodiments, at least about 90% of the drug delivery that will occur from the system occurs within the first 12 hours after application.

While not wanting to be bound by any theory, it is believed that the enhancer impacts drug delivery by at least two mechanism: by making the skin more permeable to the drug, and by solubilizing the drug so that more drug is available for delivery. When the system is in use, the amount of enhancer present in the drug-containing composition is depleted over time. Thus, over time, less enhancer is available to make the skin more permeable to the drug. Additionally, over time, less enhancer is available to solubilize the drug, which may lead to the drug crystallizing in the composition. Because only solubilized drug can be delivered transdermally, this crystallization reduces drug delivery. Thus, by selecting and controlling the type and amount of enhancer, one can select and control the drug delivery profile, such as by using an amount of a given enhancer that will result in drug delivery over a relatively short period of time, even if the system is left in place for a longer period. The impact of the amount of enhancer on drug delivery is illustrated in the examples below.

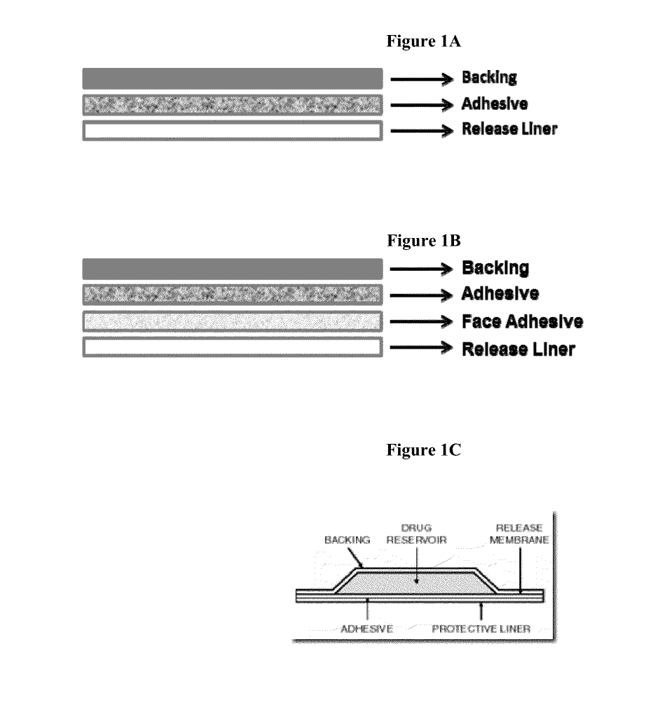

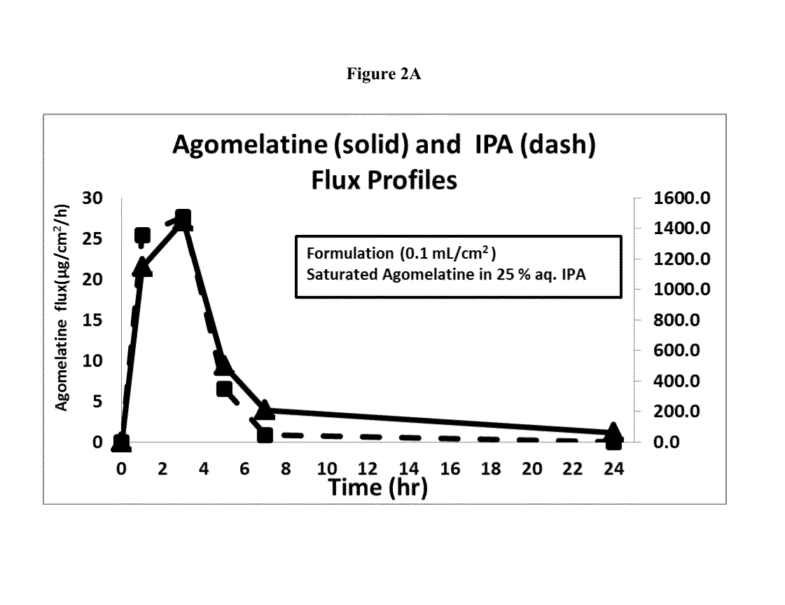

In accordance with some embodiments, the transdermal drug delivery systems described herein consist of a backing, a drug-containing composition (e.g., drug-in-solution reservoir, drug-in-gel reservoir, or drug-in-polymer matrix layer), and a release liner, as illustrated in FIG. 1A with regard to drug-in-polymer matrix embodiments, and as illustrated in FIG. 1C with regard to reservoir-type systems. In accordance with some embodiments, the transdermal drug delivery systems described herein consist of a backing, a drug-containing layer, a face adhesive, and a release liner, as illustrated in FIG. 1B. In specific embodiments, the face adhesive is a silicone face adhesive comprising a silicone adhesive, such as a silicone pressure-sensitive adhesive and, optionally, one or more penetration enhancers, such as those discussed below.

Agomelatine

Agolmelatine is a solid (a white crystalline powder) at room temperature, with a melting point of 107-109° C. It is neutral, relatively hydrophobic, and has a solubility of 0.2 mg/ml in water.

The chemical name of agolmelatine is N-[2-(7-methoxynaphthalen-1-yl)ethyl]acetamide. Its chemical structure is set forth below.

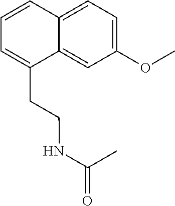 [SMILES: COc1ccc2cccc(CCNC(C)=O)c2c1]

Drug-in-Solution/Gel Reservoir Systems

In some embodiments, the transdermal drug delivery systems described herein are of the drug-in-solution or gel reservoir-type. In specific embodiments, such systems exhibit desired pharmacokinetic properties, such as drug delivery period of under 12 hours, under 8 hours, under 6 hours, or under 4 hours.

In a typical reservoir-type system, there is a separate drug reservoir and adhesive layer. Typically, a reservoir-type system is comprised of a backing layer which is sealed at its periphery to a release membrane or other layer, thus defining a drug reservoir. The skin-facing surface of the release membrane may be provided with an adhesive, or the system may have a peripheral adhesive region for affixing the system to the skin. As with matrix-type systems, the adhesive is usually covered by a release liner or protective layer which is removed before use.

In a typical reservoir-type system, the drug is formulated in a composition comprising a solvent (such as isopropanol or ethanol, or another skin-tolerable solvent), optionally with a gelling agent, and/or a non-adhesive polymer (such as cellulose derivatives, gums, silicone fluids, etc.), to form a drug-in-solution, paste-like suspension, gel, or viscous medium. Typically, the solvent(s) and/or polymer(s) are selected for biocompatibility and chemical compatibility with the drug and other components to be formulated in the system. The release membrane can be porous (permeable) or non-porous (semi-permeable) to the drug, with the latter providing rate-controlling properties. In some embodiments, the release membrane is porous, such that the drug is released by diffusion directly through the membrane material, with the membrane material having little or substantially no rate-limiting effect on the rate of passage of drug molecules. In other embodiments, wherein the membrane is non-porous membranes, the rate of passage of the drug molecules can be controlled by selection of the membrane material, e.g., its composition, thickness, and pore size. Typically, ethylene vinyl acetate (EVA), ethyl cellulose, silicon rubber and polyurethanes are used to prepare rate-controlling release membranes.

As discussed above and illustrated in the examples, the specific type(s) and relative amounts of the solution or gel reservoir components and penetration enhancer(s) can be selected and adjusted to control and modify the properties of the system, including the drug delivery profile (e.g., pharmacokinetic profile) and physical properties (e.g., tackiness, wear, etc.).

Drug-in-Polymer Matrix Systems

In some embodiments, the transdermal drug delivery systems described herein are of the drug-in-polymer matrix type. In specific embodiments, such systems exhibit desired pharmacokinetic properties, such as drug delivery period of under 12 hours, under 8 hours, under 6 hours, or under 4 hours.

In some embodiments, the transdermal drug delivery system comprises a drug-containing polymer matrix that comprises a pressure-sensitive adhesive or bioadhesive, and is adopted for direct application to a user's (e.g., a subject's) skin. In other embodiments, the polymer matrix is non-adhesive and may be provided with separate adhesion means (such as a separate adhesive layer) for application and adherence to the user's skin.

As used herein, “polymer matrix” refers to a polymer composition which contains one or more drugs. In some embodiments, the matrix comprises a pressure-sensitive adhesive polymer or a bioadhesive polymer. In other embodiments, the matrix does not comprise a pressure-sensitive adhesive or bioadhesive. As used herein, a polymer is an “adhesive” if it has the properties of an adhesive per se, or if it functions as an adhesive by the addition of tackifiers, plasticizers, crosslinking agents or other additives. Thus, in some embodiments, the polymer matrix comprises a pressure-sensitive adhesive polymer or a bioadhesive polymer, with drug dissolved or dispersed therein. The polymer matrix also may comprise tackifiers, plasticizers, crosslinking agents, enhancers, co-solvents, fillers, antioxidants, solubilizers, crystallization inhibitors, or other additives described herein. U.S. Pat. No. 6,024,976 describes polymer blends that are useful in accordance with the transdermal systems described herein. The entire contents of U.S. Pat. No. 6,024,976 is incorporated herein by reference.

As used herein, the term “pressure-sensitive adhesive” refers to a viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

In some embodiments, the polymer matrix is a pressure-sensitive adhesive at room temperature and has other desirable characteristics for adhesives used in the transdermal drug delivery art. Such characteristics include good adherence to skin, ability to be peeled or otherwise removed without substantial trauma to the skin, retention of tack with aging, etc. In some embodiments, the polymer matrix has a glass transition temperature (Tg), measured using a differential scanning calorimeter, of between about −70° C. and 0° C.

As used herein, the term “rubber-based pressure-sensitive adhesive” refers to a viscoelastic material which has the properties of a pressure-sensitive adhesive and which contains at least one natural or synthetic elastomeric polymer.

In some embodiments, the transdermal drug delivery system includes one or more additional layers, such as one or more additional polymer matrix layers, or one or more adhesive layers that adhere the transdermal drug delivery system to the user's skin, such as a face adhesive layer. In other embodiments, the transdermal drug delivery system is monolithic, meaning that it comprises a single polymer matrix layer comprising a pressure-sensitive adhesive or bioadhesive with drug dissolved or dispersed therein, and no rate-controlling membrane or other polymeric adhesive layer. As used herein, a “monolithic” transdermal drug delivery system may include a backing layer and/or release liner, and may be provided in a package.

As noted above, in a drug-in-polymer matrix system, the drug is formulated in a polymer matrix, such as a pressure-sensitive adhesive polymer matrix, optionally with a penetration enhancer. In embodiments where the polymer matrix is an adhesive, the polymer matrix serves both as the means for affixing the patch to the skin and as the carrier for the drug. In a monolithic polymer matrix device, the drug-in-polymer matrix layer is sandwiched between a backing and a release liner. In use, the release liner is removed, and the drug-in-adhesive polymer matrix layer is applied directly onto the skin. In accordance with any polymer matrix embodiments, any polymer matrix suitable for use as the polymer matrix of a transdermal drug delivery system can be used, such as rubber-based polymers and pressure-sensitive adhesives, such as acrylic, silicone, polyisobutylene and styrene-isoprene-styrene polymers and pressure-sensitive adhesives known in the art or developed for use in transdermal drug delivery systems, including mixtures and blends thereof.

In embodiments comprising a penetration enhancer, the polymer components and penetration enhancer are selected so that the penetration enhancer is miscible with the polymer matrix components, and so that the penetration enhancer is capable of being formulated in the polymer matrix components while still providing a system with acceptable physical properties for adhering the system to the skin of the subject during use.

In some embodiments the polymer components and penetration enhancer are selected so that the penetration enhancer can be the only processing solvent for the polymer components. Taking isopropanol as an exemplary penetration enhancer, suitable polymers include acrylic polymers such as Duro-Tak 87-900A (which includes non-reactive amide groups), Duro-Tak 87-202A (which includes cross-linked carboxylic groups), Duro-Tak 87-2979 (which include cross-linked carboxylic/hydroxyl groups), Duro-Tak 387-4287 (which includes hydroxyl groups), all sold by Henkel Corporation, Bridgewater, N.J., and silicone polymers such as Bio-PSA 4502, and Bio-PSA 4402, sold by Dow Corning Corporation, Medical Products, Midland, Mich.

The specific type(s) and relative amounts of polymer matrix components and penetration enhancer(s) can be selected and adjusted to control and modify the properties of the system, including the drug delivery profile (e.g., pharmacokinetic profile) and physical properties (e.g., adhesion, tackiness, wear, etc.).

Penetration Enhancer

As noted above, in some embodiments, the transdermal drug delivery systems described herein include a penetration enhancer. In some embodiments, the penetration enhancer is a concentration-dependent skin permeation enhancer, such as isopropanol (“IPA”) or ethanol.

A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, the penetration enhancer comprises a mixture of at least two penetration enhancers.

As noted above, penetration enhancers that are particularly suitable in the systems described herein include but are not limited to concentration-dependent skin permeation enhancers, such as isopropanol (“IPA”) and ethanol.

In some embodiments of polymer matrix-type systems, a penetration enhancer is used in an amount up to about 35% by dry weight of the polymer matrix, including up to 30% by weight, up to about 20% by weight, including 20% by weight, or up to about 10% by weight, up to 10% by weight, or up to 5% by weight, including up to 5% by weight, based on the dry weight of the polymer matrix. In some embodiments, a penetration enhancer is used in an amount of from about 5% to about 20%, including about 10% by weight. In some embodiments of reservoir-type systems, a greater amount of penetration enhancer is used, such as up to 40%, up to 50%, up to 60%, up to 70%, up to 75%, or up to 80%.

Acrylic Polymers

In some embodiments, the transdermal drug delivery system comprises a polymer matrix that comprises an acrylic polymer. The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups. Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups.

In some embodiments, the acrylic polymer includes hydroxy functional monomers. Such polymers generally exhibit good solubility for norelgestromin, which allows sufficient loading of norelgestromin for preparation of a system that achieves transdermal delivery of a therapeutically effective amount of active agent over an extended period of time, such as a period of at least 3 days, at least 4 days, or at least 7 days, or longer.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

Suitable acrylic polymers also include pressure-sensitive adhesives which are commercially available, such as the acrylic-based adhesives sold under the trademarks DURO-TAK® (such as DURO-TAK® 87-900A, 87-2516, 87-2287, -4098, -2852, -2196, -2296, -2194, -2516, -2070, -2353, -2154, -2510, -9085, -9088 and 73-9301) and GELVA® Multipolymer Solution (such as GELVA® 2480, 788, 737, 263, 1430, 1753, 1151, 2450, 2495, 3067, 3071, 3087 and 3235) both by Henkel Corporation, Bridgewater, N.J. Other suitable acrylic adhesives include those sold under the trademark EUDRAGIT® by Evonik Industries AG Pharma Polymers, Darmstadt, Germany. For example, hydroxy functional adhesives with a reactive functional OH group in the polymeric chain can be used. Non-limiting commercial examples of this type of adhesive includes both GELVA® 737, 788, and 1151, and DURO-TAK® 87-2287, -4287, -2510 and -2516.

Silicone Polymers

In some embodiments, the transdermal drug delivery system comprises a polymer matrix that comprises a silicone polymer and/or comprises a face adhesive that comprises a silicone polymer. The term “silicone polymer” is used interchangeably with the terms silicon polymers, siloxane, polysiloxane, and silicones as used herein and as known in the art. Suitable silicone polymers include silicone pressure-sensitive adhesives. Thus, in some embodiments, the silicone polymer is an adhesive polymer, such as a pressure-sensitive adhesive. In other embodiments, the silicone polymer functions as an adhesive by the addition of one or more tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al. “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series, such as -4502, and -4600 series.

Further details and examples of silicone pressure-sensitive adhesives which are useful in the polymer matrices and compositions and methods described herein are mentioned in the following U.S. Pat. Nos. 4,591,622; 4,584,355; 4,585,836; and 4,655,767, which are all expressly incorporated by reference herein in their entireties. It should also be understood that silicone fluids are also contemplated for use in the polymer matrices and methods described herein.

Polyisobutylene Polymers

In some embodiments, the transdermal drug delivery system comprises a polymer matrix that comprises a polyisobutylene polymer. Polyisobutylene polymers suitable for use in polymer matrix compositions are known and are available commercially, and include those sold by BASF under the Oppanol® B brand, which is a series of medium and high molecular weight polyisobutylene polymers having a weight-average molecular weight (MW) between 40,000 and 4,000,000, and include Oppanol® B100 and Oppanol® B11SFN. In some embodiments, the polymer matrix comprises two or more polyisobutylene polymers of different molecular weights. In accordance with these embodiments, the relative amounts of polyisobutylene polymers can be selected and tailored to produce a product with satisfactory physical and pharmacokinetic properties.

In some embodiments where the polymer matrix comprises one or more polyisobutylene polymers, the polymer matrix also includes one or more tackifiers. Suitable tackifiers for use with PIB polymers in transdermal drug delivery systems are known in the art and include hydrocarbon resins, mineral oil, and hydrogenated polyisobutenes, such as Panalane® sold by Lipo Chemicals, Inc. (Paterson, N.J.). In some embodiments, the tackifier is a hydrogenated polyisobutenes, such as Panalane®. In some embodiments, the tackifier is a hydrogenated hydrocarbon resin. In some embodiments, a polyisobutylene matrix include an acrylic polymer that acts as a tackifier, such as one or more of those discussed below (e.g., as DURO-TAK® 87-900A). Such systems optionally may further comprise one or more modifiers such a silicone fluid (e.g., cyclomethicone) and SiO₂ or TiO₂, such as may be useful to improve cohesions (shear value) and/or decrease cold flow.

In some embodiments, the transdermal drug delivery system comprises a peripheral adhesive that comprises a polyisobutylene polymer or polymer composition as discussed above.

Styrene-Isoprene-Styrene Polymers

In some embodiments, the transdermal drug delivery system comprises a polymer matrix that comprises a styrene-isoprene-styrene block copolymers (SIS polymers). Styrene-isoprene-styrene block copolymers suitable for use in polymer matrix compositions are known and are available commercially, and include those sold by Kraton Polymers US under the Kraton® brand name, such as Kraton® D1111 KT.

In some embodiments where the polymer matrix comprises one or more SIS polymers, the polymer matrix also includes one or more tackifiers. Suitable tackifiers for use with SIS polymers in transdermal drug delivery systems are known in the art and include hydrocarbon resins and other pressure-sensitive adhesives, such as acrylic pressure-sensitive adhesives and silicone pressure-sensitive adhesives, such as any of those discussed above. In some embodiments, the tackifier is a hydrogenated hydrocarbon resin, such as Arkon P-100 (Arakawa Chemical Industries, Osaka, Japan).

In some embodiments, the transdermal drug delivery system comprises a peripheral adhesive that comprises an SIS polymer or polymer composition as discussed above.

Antioxidants

In some embodiments, the drug-containing composition (e.g., solution, gel or polymer matrix) comprises an antioxidant. In some embodiments, the antioxidant is butylhydroxytoluene (BHT) and/or butylhydroxyanisole (BHA). In other embodiments, the antioxidant is, additionally or alternatively, alpha tocopherol, ascorbic-acid, ascorbyl palmitate, propyl gallate, fumaric acid, malic acid, sodium ascorbate, sodium metabisulfite, and the like. In specific embodiments, the antioxidant (or combinations thereof) are used in a total amount of from about 0.01 to about 5.0% by weight, including from about 0.01 to about 0.1%, such as about 0.05% by weight, or from about 0.1 to about 1.0%, such as about 0.1% by weight, about 0.25% by weight, and about 0.5% by weight, based on the dry weight of the polymer matrix.

Other Components

In some embodiments, the drug-containing composition further comprises one or more thickeners, fillers, and/or other additives or components known for use in transdermal drug delivery systems.

Transdermal Delivery Systems

The transdermal delivery systems described herein may be of any shape or size suitable for transdermal application.

The drug-in-polymer matrices described herein may be prepared by methods known in the art. For example, the polymer matrix material can be applied to a backing layer and release liner by methods known in the art, and formed into sizes and shapes suitable for use. For example, after the polymer matrix is formed, it may be brought into contact with a support layer, such as a releaser liner layer or backing layer, in any manner known to those of skill in the art. Such techniques include calender coating, hot melt coating, solution coating, etc.

For example, a polymer matrix can be prepared by blending the components of the polymer matrix in the presence of a solvent, such as a volatile organic solvent, applying the wet blend of matrix material to a support layer such as a backing layer or release liner, removing any remaining solvents, and laminating to a release line or backing layer. The drug can be added at any stage. In one embodiment, all polymer matrix components, including drug, are blended together. In another embodiment, the polymer matrix components other than drug are blended together, and then the drug is dissolved or dispersed therein. The order of steps, amount of ingredients, and the amount and time of agitation or mixing can be determined and optimized by the skilled practitioner. Exemplary methods are illustrated in the examples.

Individual units can be cut from a laminate produced as described above and packaged in a pouchstock, as discussed above.

The reservoir-type systems described herein may be prepared by methods known in the art. For example, as discussed above, a reservoir space is formed between a backing material and a release membrane material, filled with the drug-containing composition (prepared as described above), and sealed. The skin-facing side of the release membrane is provided with a pressure-sensitive adhesive, or the system is provided with a peripheral adhesive, with a release liner protecting the adhesive until use.

Therapeutic Methods

In some embodiments, there is provided a method of effecting transdermal drug delivery of agomelatine over delivery period of less than about 12 hours, or less than about 8 hours, by applying a transdermal drug delivery system as described herein to the skin or mucosa of a subject in need thereof. In some embodiments, the system is applied over a period of at least about 1 day, but the drug delivery occurs over a short time period, such as less than about 12 hours, or less than about 8 hours. That is, in some embodiments, drug delivery is substantially completed within less than about 12 hours, less than about 8 hours, less than about 6 hours, less than about 4 hours, or less, even if the system remains applied to the subject. As used herein the term “substantially completed” means that at least 60% of the drug that will be delivered from the system has been delivered. In specific embodiments, at least about 60% of the drug delivery that will occur from the system occurs within the first 4 to 8 hours, or 4 to 6 hours, after application. In other embodiments, at least about 75% of the drug delivery that will occur from the system occurs within the first 4 to 8 hours, or 4 to 6 hours, after application. In yet other embodiments, at least about 80% of the drug delivery that will occur from the system occurs within the first 4 to 6 hours after application. In still other embodiments, at least about 80% of the drug delivery that will occur from the system occurs within the first 6 to 12 hours, or 6 to 8 hours, after application. In still other embodiments, at least about 90% of the drug delivery that will occur from the system occurs within the first 12 hours after application. In some embodiments, the method is effective to achieve therapeutic levels of agomelatine in the subject during the delivery period.

In some embodiments, the systems described herein are designed for use by subjects suffering from depression, including major depressive disorder. In specific embodiments, the systems described herein are designed for once daily use for the treatment of depression, including major depressive disorder.

The transdermal drug delivery systems described herein can be used to provide transdermal delivery of agomelatine to a subject in need thereof, such as a subject suffering from depression or major depressive disorder, by applying the system to the skin or mucosa of the subject. Thus, the transdermal drug delivery systems described herein can be used in methods of treating depression in a subject in need thereof. In specific embodiments, the methods involve applying the system once daily to the skin or mucosa of a subject in need thereof. In some embodiments, the delivery of agomelatine is substantially completed within 12 hours or less, even if the system is left in place for longer, such as for 24 hours or longer.

The following specific examples are included as illustrative of the transdermal drug delivery systems described herein. These examples are in no way intended to limit the scope of the invention. Other aspects of the invention will be apparent to those skilled in the art to which the invention pertains.

### Example 1

The following example illustrates the concentration-dependent effects of isopropanol as a penetration enhancer for agomelatine, and the short drug delivery period and “on-and-off” effect achieved with a composition as described herein, as assessed in vitro across human cadaver skin.

The solubility of agomelatine in water or different concentrations of aqueous isopropanol (IPA) was assessed. As shown in the table below, the solubility of agomelatine in aqueous IPA solutions increased with increasing IPA content. Agomelatine was formulated at its saturation concentration in different concentrations of aqueous IPA and drug flux through human cadaver skin was assessed using a modified Franz-cell apparatus, using 0.1 mL solution per cm². As shown in the table below, maximum drug flux was observed at 50-75% IPA, with drug flux falling off at 90% IPA.

| Aqueous | Agomelatine | Relative Flux Through Human Skin |
|---|---|---|
| IPA | Solubility @ 32° C. | From Saturated Agomelatine @32° C. |
| (v/v %) | (mg/mL) | (μg/cm²/hr) |
| 0 (water) | 0.4 | 1 (reference) |
| 25 | 3 | 2-11 |
| 50 | 51 | 5-17 |
| 75 | 139 | 4-15 |
| 90 | >150 | 2-5 |

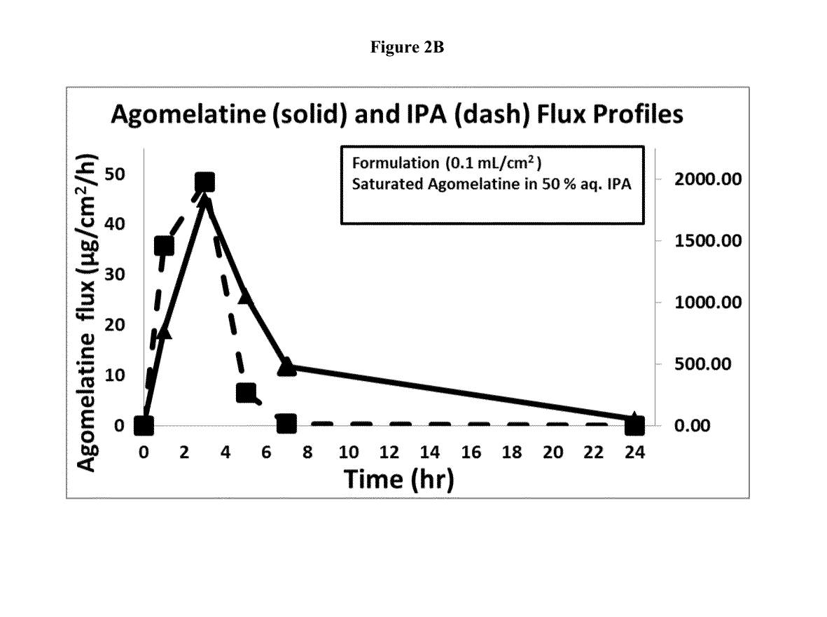

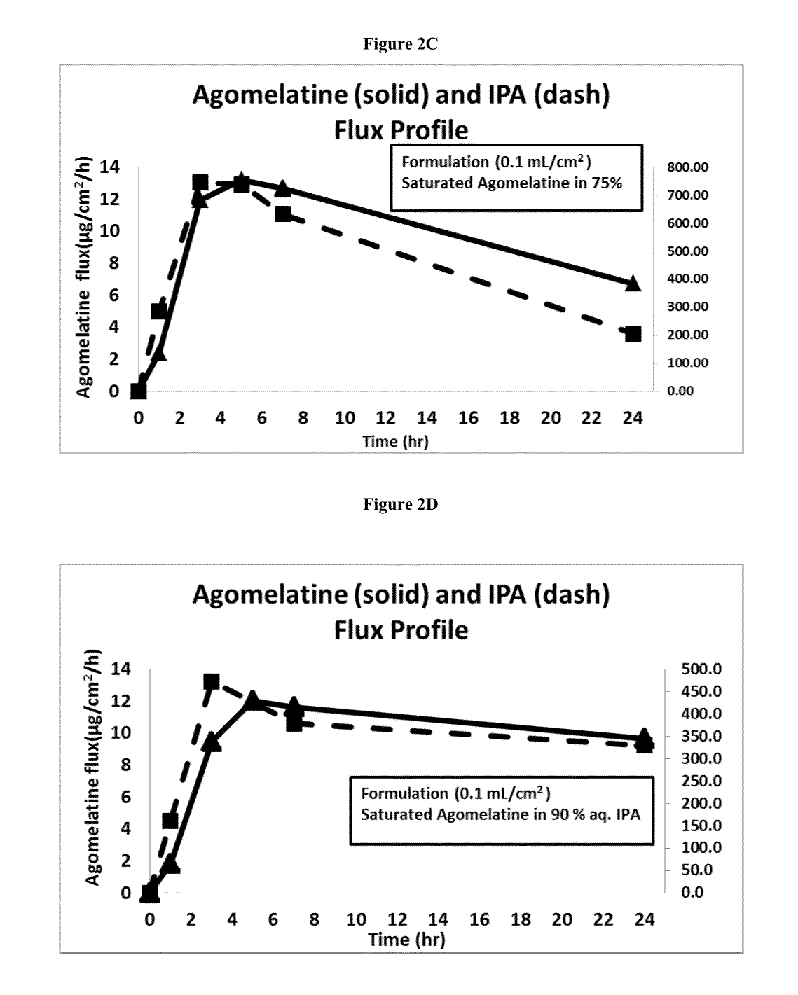

FIGS. 2A-2D show the flux of agomelatine and IPA from the 25%, 50%, 75% and 90% IPA compositions, respectively. As shown in the figures, changing the amount of IPA impacts the drug delivery profile. The total amount of drug delivered from the 50% IPA composition was about 180 g/cm², indicating that this solution could be used in a 7 or 14 cm² system to provide a dose equivalent to an oral dose of 25 or 50 mg, respectively. (Current commercial oral dosage achieve the systemic delivery of 1.25 to 2.5 mg agomelatine in humans after hepatic metabolism).

### Example 2

The following example illustrates the short term drug delivery and “on-and-off” effect achieved with a composition as described herein, as assessed in vivo in swine.

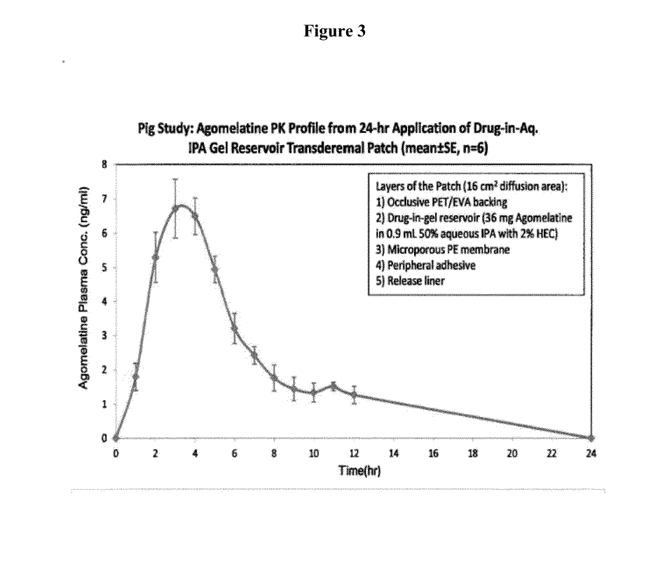

Transdermal drug delivery systems of the drug-in-gel reservoir type having a 16 cm² active surface area were prepared using a gel reservoir comprising 36 mg agomelatine in 0.9 mL aqueous IPA (50% v/v) and 2% hydroxyethylcellulose (HEC). The systems included an occlusive polyester/ethylene vinyl acetate (PET/EVA) backing, a microporous polyethylene (PE) membrane disposed between the gel reservoir and a release liner, and peripheral adhesive for adhering the systems to the skin. The system were applied to the skin of live swine for 24 hours, and blood samples were drawn periodically to assess plasma levels of agomelatine and IPA. Results are shown in FIG. 3. As shown in the figure, the transdermal drug delivery systems achieved a short drug delivery period with an “on-and-off” effect, with substantially all of the agomelatine delivery occurring within 5-6 hours, even though the systems were left in place for 24 hours. The systems delivered a total of about 3.8 mg agomelatine.

### Example 3

The following example illustrates embodiments using a drug-in-polymer matrix type system and the short drug delivery period and “on-and-off” effect achieved with a composition as described herein, as assessed in vitro across human cadaver skin.

| | Maximum IPA | Agomelatine |
|---|---|---|
| | % w/w To Retain | Solubility |
| Polymer(s) | good Adhesion | % w/w |
| Duro-Tak 387-4287 | 4.5 | <12.5 |
| Duro-Tak 87-2979 | 4.2 | <15.0 |
| Duro-Tak 87-202A | 4.3 | <12.5 |
| Duro-Tak 87-900A | 6.1 | <12.5 |
| Duro-Tak 387-4287 (72% w/w dry) + | 6.1 | 10.5 |
| Bio-PSA 4402 (28% w/w dry) | | |

When silicone polymers were used as the only polymer component and formulated with IPA and agomelatine, it was difficult to obtain a polymer matrix with good physical properties.

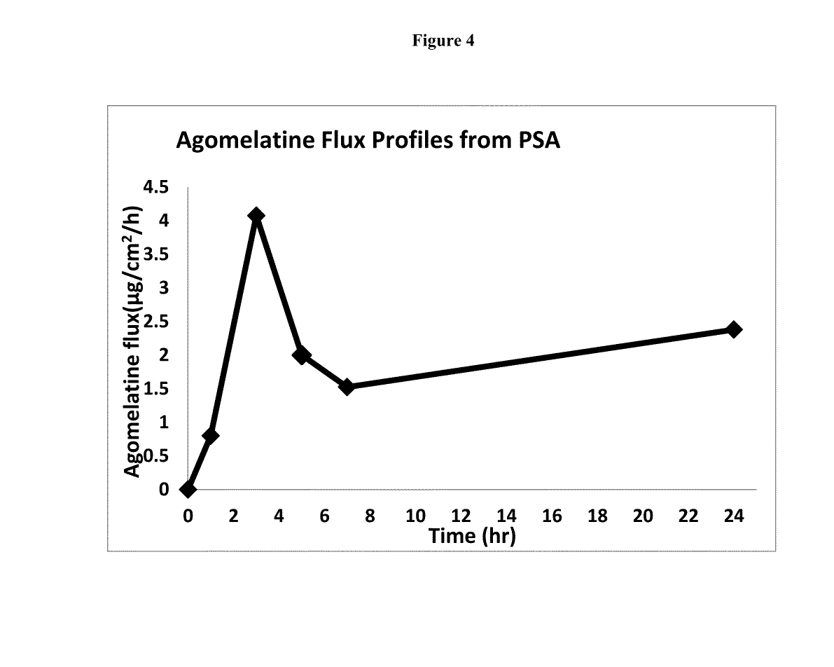

Drug-in-polymer matrix type systems were prepared using polymer components comprised of 72% Duro-Tak 387-4287 and 28% Bio-PSA 4402 (on a dry wt/wt basis), formulated with 6.1% w/w IPA and 10.5% w/w agomelatine. Drug flux through human cadaver skin was assessed using a modified Franz-cell apparatus. Results are shown in FIG. 4. As shown in the figure, the transdermal drug delivery systems achieved a short drug delivery period with an “on-and-off” effect, with substantially all of the agomelatine delivery occurring within 5-6 hours, even though the systems were left in place for 24 hours.

What is claimed is:

1. A transdermal drug delivery system for the transdermal delivery of agomelatine in the form of a flexible, finite system, comprising a composition comprising agomelatine and an enhancer.

2. The transdermal drug delivery system of claim 1, wherein the enhancer is selected from the group consisting of isopropanol and ethanol.

3. The transdermal drug delivery system of claim 1, wherein the composition comprises an amount of the enhancer effective to achieve substantially complete drug delivery within 12 hours of application or less.

4. The transdermal drug delivery system of claim 1, wherein the composition comprises an amount of the enhancer effective to achieve at least about 60% of the agomelatine delivery within about 4 to about 6 hours of application.

5. The transdermal drug delivery system of claim 1, wherein the composition comprises an amount of the enhancer effective to achieve at least about 75% of the agomelatine delivery within about 4 to about 6 hours of application.

6. The transdermal drug delivery system of claim 1, wherein the composition comprises an amount of the enhancer effective to achieve at least about 80% of the agomelatine delivery within about 6 to about 8 hours of application.

7. The transdermal drug delivery system of claim 1, wherein the composition is a drug-in-solution reservoir-type composition comprising agomelatine and aqueous isopropanol.

8. The transdermal drug delivery system of claim 7, wherein the aqueous isopropanol comprises 50-75% v/v isopropanol.

9. The transdermal drug delivery system of claim 7, wherein the agomelatine is present at its saturation concentration.

10. The transdermal drug delivery system of claim 7, wherein the composition comprises at least 50 mg/mL agomelatine.

11. The transdermal drug delivery system of claim 1, wherein the composition is a drug-in-gel reservoir-type composition comprising agomelatine, aqueous isopropanol, and a gelling agent.

12. The transdermal drug delivery system of claim 11, wherein the aqueous isopropanol comprises 50-75% v/v isopropanol.

13. The transdermal drug delivery system of claim 11, wherein the agomelatine is present at its saturation concentration.

14. The transdermal drug delivery system of claim 11, wherein the composition comprises at least 50 mg/mL agomelatine.

15. The transdermal drug delivery system of claim 1, wherein the composition is a drug-in-polymer matrix composition comprising agomelatine formulated in a polymer matrix comprising isopropanol.

16. The transdermal drug delivery system of claim 15, wherein the polymer matrix comprises a pressure sensitive adhesive polymer.

17. The transdermal drug delivery system of claim 16, wherein the polymer matrix comprises an acrylic polymer, a silicone polymer, or a mixture of two or more thereof.

18. The transdermal drug delivery system of claim 16, wherein the polymer components comprise about 70% (w/w) acrylic polymer and about 30% (w/w) silicone polymer, based on the dry weight of the polymer components.

19. The transdermal drug delivery system of claim 15, wherein the agomelatine is present at its saturation concentration in the polymer matrix.

20. The transdermal drug delivery system of claim 15, wherein the polymer matrix composition comprises about 10.5% (w/w) agomelatine and about 6% (w/w) isopropanol.

21. The transdermal drug delivery system of claim 1, further comprising a backing.

22. The transdermal drug delivery system of claim 1, further comprising a release liner.

23. A method of making a transdermal drug delivery system for the transdermal delivery of agomelatine, comprising preparing a drug-containing composition comprising agomelatine and an enhancer.

24. A method of transdermally delivering agomelatine, comprising applying a transdermal drug delivery system according to claim 1 to the skin or mucosa of a subject in need thereof.

25. A method of treating depression in a subject in need thereof, comprising applying a transdermal drug delivery system according to claim 1 once daily to the skin or mucosa of a subject in need thereof.
